(19) United States
(12) Patent Application Publication
(10) Pub. No.: US 2010/0022767 A1
(43) Pub. Date: Jan. 28, 2010

(54) DEVELOPMENT OF A SYNTHESIS OF SYRINGOLIN A AND B AND DERIVATIVES THEREOF

(75) Inventors: Markus Kaiser, Herdecke (DE); Jérôme Clerc, Dortmund (DE)

Correspondence Address: Gregory A. Nelson;Novak Druce & Quigg LLP, 525 Okeechobee Blvd, Suite 1500, West Palm Beach, FL, 33401, US

(73) Assignee: MAX-PLANCK-GESELLSCHAFT ZUR FORDERUNG DER WISSENSCHAFTEN E.V., Munich (DE)

(21) Appl. No.: 12/508,964

(22) Filed: Jul. 24, 2009

Related U.S. Application Data
(60) Provisional application No. 61/129,865, filed on Jul. 25, 2008.

(51) Int. Cl. C07D 245/02

(52) U.S. Cl. 540/460

(57) ABSTRACT

The synthesis of syringolin A and B and derivatives thereof as well as to pharmaceutical compositions containing the syringolin A or B or derivatives thereof and the use of syringolin A and B and derivatives thereof for prophylaxis and treatment of cancer.

### CROSS REFERENCE TO RELATED APPLICATIONS

This application claims the priority of U.S. Provisional Patent Application No. 61/129,865, filed Jul. 25, 2008, which is incorporated herein by reference in its entirety.

### SUMMARY OF THE INVENTION

The present invention relates to a synthesis of syringolin A and B and derivatives thereof as well as to pharmaceutical compositions containing said syringolin A or B or derivatives thereof and the use of syringolin A and B and derivatives thereof for prophylaxis and treatment of cancer.

### BACKGROUND OF THE INVENTION

Syringolin A and syringolin B are plant elicitors produced by the plant pathogen Pseudomonas syringae. Both syringolin A and syringolin B are potent irreversible inhibitors of proteasome. Proteasome inhibitors are compounds that block the action of proteasomes, cellular complexes that break down proteins, like the p53 protein. Proteasome inhibitors are known to be suitable pharmaceutically active compounds for the treatment of proliferative diseases such as cancer.

Syringolin A is an unusual secreted peptide consisting of a 12-membered ring formed by the two non-proteinogenic amino acids 5-methyl-4-amino-2-hexenoic acid and 3,4-dehydrolysine. The α-amino group of the latter is connected by a peptide bond to a valine that in turn is linked to a second valine via a urea moiety.

The chemical formula of syringolin A is as follows:

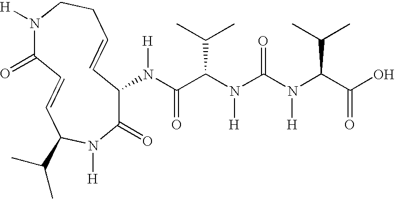

The chemical formula of syringolin B is as follows:

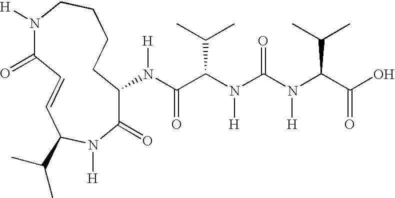

It is the object of the present invention to provide a synthetic route for preparing syringolin A and syringolin B as well as derivatives of syringolin A and syringolin B.

The object of the present invention is solved by the teaching of the independent claims. Further advantageous features, aspects and details of the invention are evident from the dependent claims, the description, and the examples of the present application.

### DETAILED DESCRIPTION

The present invention relates to the full chemical synthesis of syringolin A and syringolin B according to the synthetic route disclosed in detail in the following.

### Chemical Synthesis of Syringolin A

The chemical synthesis starts from the protected amino acid valin. The Boc (tert-butoxycarbonyl) protecting group is preferred for the amino function and an ester protecting group such as a methyl ester is preferred for the carboxylic acid group.

Thus the chemical synthesis starts with compound A wherein PG and PG′ refer independently of each other to a suitable protecting group.

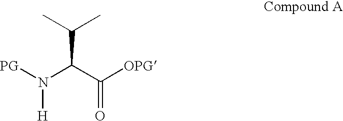

Compound A is reacted at low temperatures preferably below −50° C. and more preferably below −70° C. with a strong base such as DIBAL-H and thereafter with Ph₃P═COOPG″ in a suitable solvent such as methylenchloride (DCM), tetrahydrofurane (THF), chloroform or the like in order to obtain compound B. In Ph₃P═COOPG″ the group PG″ refers to a suitable protecting group such as methyl (Me). PG refers preferably to a Boc protecting group.

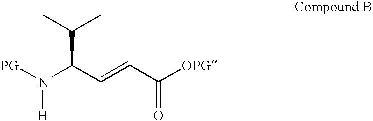

Compound B is reacted with osmium tetroxide (OsO₄), sodium periodate (NalO₄) and N-methylmorpholine-N-oxide (NMO) in acetone/water (2:1) and a ketal ring is subsequently formed using 2,2-dimethoxypropane (2,2-DMP) and pyridinium p-toluene sulfonate (PPTS) in DCM under reflux in order to obtain compound C.

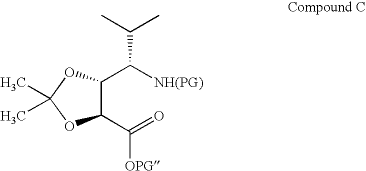

Compound C is deprotected under basic conditions using for example lithium hydroxide (LiOH) in methanol/water mixture and is subsequently reacted with 1-aminobut-3-en hydrochloride preferably in presence of PyBOP (benzotriazol-1-yl-oxytripyrrolidinophosphonium hexafluorophosphate), DIEA (N,N-diisopropylethyl-amine) and 1-hydoxybenzotriazole (HOBt) in a polar aprotic solvent such as methylene chloride (DCM) in order to obtain compound D.

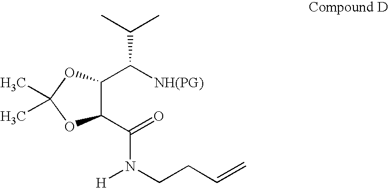

Compound D is than deprotected for instance by means of 2,6-lutidine and trimethylsilyl trifluoromethanesulfonate (TMSOTf) in a polar aprotic solvent such as DCM, THF, chloroform.

Thereafter compound E wherein PG** refers to a suitable amino protecting group such as Boc

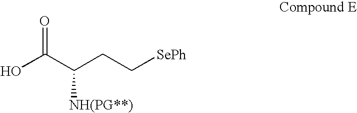

is added together with PyBOP, DIEA and 1-hydroxy-7-azabenzotriazole (HOAt) in a polar aprotic solvent such as DCM to generate compound F.

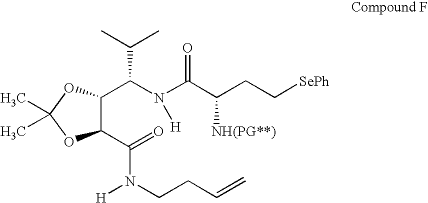

Thereafter an oxidation step with hydrogen peroxide is performed preferably in the presence of DIEA and in a polar aprotic solvent such as DCM in order to produce compound G.

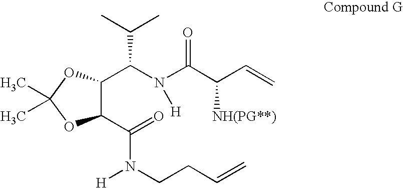

Now as key step a macrolactamisation is carried out using the Grubbs II catalyst (Sigma-Aldrich, catalog No. 569747-2g) preferably in toluene and at elevated temperatures preferably between 80° C. and 100° C. to obtain compound H

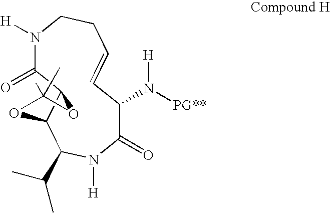

After a deprotection step with for instance 2,6-lutidine and TMSOTf in a polar aprotic solvent such as DCM, THF, chloroform the obtained deprotected product is reacted with compound J

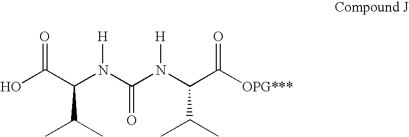

wherein PG*** refers to a suitable ester protecting group preferably in the presence PyBOP, DIEA in DCM in order to obtain compound K.

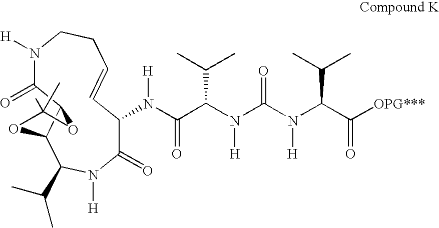

Now the ketal group of compound K is cleaved with an organic acid such as formic acid in water/THF and thereafter the vicinal dihydroxy group is converted to a thiocarbonate group be use of CS(lm)₂ and 4-(dimethylamino)-pyridine (DMAP) in a polar aprotic solvent such as THF to obtain compound L.

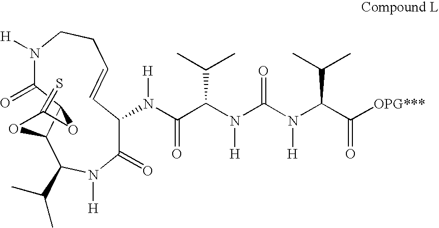

The thiocarbonate group of compound L is converted to a double bond using P(OMe)₃ and subsequently the ester protecting group PG*** is cleaved under basic conditions, for instance, with LiOH in methanol/water to result in the final product syringolin A.

### Chemical Synthesis of Syringolin B

The chemical synthesis starts from the protected amino acid valin. The Boc (tert-butoxycarbonyl) protecting group is preferred for the amino function and an ester protecting group is preferred for the carboxylic acid group.

Thus the chemical synthesis starts with compound 1 wherein PG and PG′ refer independently of each other to a suitable protecting group.

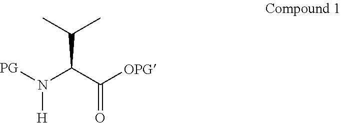

Compound 1 is reacted at low temperatures preferably below −50° C. and more preferably below −70° C. with a strong base such as DIBAL-H and thereafter with Ph₃P═COOPG″ in a suitable solvent such as methylenchloride (DCM), tetrahydrofurane (THF), chloroform or the like in order to obtain compound 2. In Ph₃P═COOPG″ the group PG″ refers to a suitable protecting group such tert-butyl (tBu).

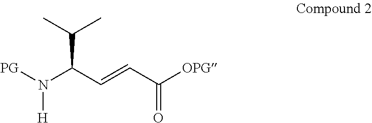

Compound 2 is reacted at temperatures from −20° C. to room temperature and preferably at temperatures from −5° C. to 10° C. for about one day with hydrochlorid acid and PG″OAc in a suitable solvent such as dioxane. PG″ in PG″OAc has the same meaning as PG″ in compound 2.

Thereafter the deprotected compound is successively converted to compound 3 by conversion with PG′″-Lys(PG″″)-OH in a polar aprotic solvent such as DCM, THF, chloroform. PG′″ refers to a suitable amino protecting group such as Boc and PG″″ refers to another amino protecting group such as Troc (2,2,2-trichloroethoxycarbonyl). Furthermore it is preferred if further chemical substances such as PyBOP (benzotriazol-1-yl-oxytripyrrolidinophosphonium hexafluorophosphate) or DIEA (N,N-diisopropylethylamine) are added.

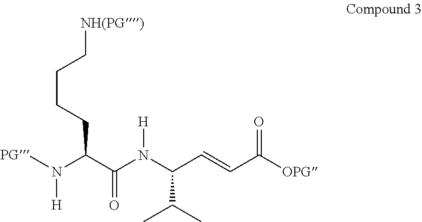

Next, compound 3 is reacted at temperatures from −20° C. to room temperature and preferably at temperatures from −5° C. to 10° C. for about one day with hydrochlorid acid and PG″OAc in a suitable solvent such as dioxane. PG″ in PG″OAc has the same meaning as PG″ in compound 3.

After the deprotection step the obtained product is subsequently converted to compound 5 by reaction with compound 4 in a polar aprotic solvent such as DCM, THF, CHCl₃.

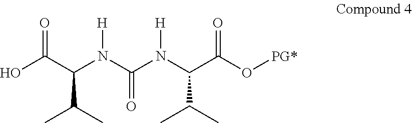

wherein PG* represents a suitable carboxy protecting group such as Fm (fluorenylmethyl). Moreover it is preferred to use further additives in the reaction such as PyBOP and/or DIEA.

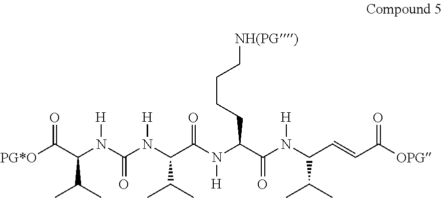

Compound 5 is deprotected in a first deprotection step in a solution of an organic acid such as acetic acid in an organic solvent such as THF preferably under the presence of zinc and in a second deprotection step in an aqueous solution of a suitable acid such as formic acid, hydrochlorid acid or mixtures thereof. Compound 6 is obtained in almost quantitative yield.

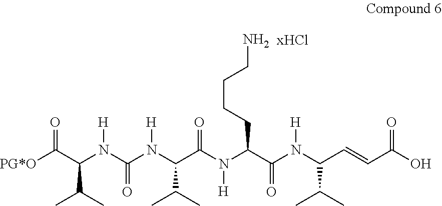

Finally a macrolactamisation reaction is carried out using PyBOP, acetic acid, DIEA in DMF for one to 3 days, preferably 2 days followed by a final deprotection step using a base such as piperidine in a suitable solvent preferably the solvent already used for the macrolactamisation. The final product syringolin B is obtained in an overall yield of about 7%.

### Examples

### Example 1

### Synthesis of Boc and CH₃ Protected Compound C

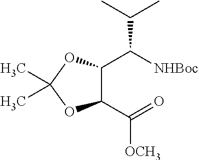

Compound A is commercially available from Sigma-Aldrich.

N-(tert-Butoxycarbonyl)-(L)-valine methyl ester (5.64 g, 24.39 mmol, 1 eq.) was dissolved under argon in toluene (245 mL) in a 500 mL flame-dried flask. The solution was cooled to −78° C. and a 1 M solution of DIBAL-H in toluene (49 mL, 48.78 mmol, 2 eq.) was slowly added over 2 hours. After stirring for further 2 hours, the mixture was quenched with a 1.2 M solution of potassium sodium tartrate (150 mL) and stirred vigorously at room temperature for 2 hours. The resulting solution was extracted with dichloromethane and the organic layers were dried over Na₂SO₄, filtered and concentrated to dryness to give N-(tert-butoxycarbonyl)-(L)-valinal which was directly used in the next step without further purification.

Crude N-(tert-butoxycarbonyl)-(L)-valinal was dissolved in dichloromethane (245 mL) and (methoxycarbonylmethylene)triphenylphosphorane (9.38 g, 28.05 mmol, 1.15 eq.) was added in one portion. After stirring for 12 hours, the mixture was successively washed with a 10% aq. KHSO₄ solution, a 5% aq. NaHCO₃ solution and with brine. The organic layer was dried over Na₂SO₄, filtered and evaporated to dryness. The resulting crude product was purified by flash column chromatography (10% ethyl acetate in cyclohexane) to afford 3.78 g (14.69 mmol, 60%) of this intermediate as colorless crystals.

This intermediate (643 mg, 2.50 mmol, 1 eq.) was dissolved in acetone/water (2:1, 22.5 mL) in a 100 mL flask. 4-Methylmorpholine N-oxide (440 mg, 3.75 mmol, 1.5 eq.) and osmium tetroxide solution (4% wt/H₂O, 764 μL, 125 μmol, 0.05 eq.) were added consecutively. The flask was flushed with argon and the reaction was stirred for 2 days. The reaction was quenched by addition of a saturated aq. NaHSO₃ solution and the acetone was evaporated in vacuo. Ethyl acetate and further water were added, separated in a funnel, and the organic layer was dried over Na₂SO₄, filtered over Celite and concentrated to dryness to give a crude mixture of diastereoisomers. The pure intermediate was obtained by recrystallization from cyclohexane to yield 583 mg (2.00 mmol, 80%) of a pure single diastereoisomer as colorless crystals. The residual mixture was then purified by flash column chromatography (70% diethyl ether in petroleum ether) to afford another 38 mg (0.13 mmol, 5%) as colorless crystals (=overall yield of 85%).

This intermediate (3.53 g, 12.12 mmol, 1 eq.) was then dissolved in dichloromethane (45 mL) in a 250 mL flame-dried flask and 2,2-dimethoxypropane (45 mL, 364.00 mmol, 30 eq.) and pyridinium p-toluenesulfonate (153 mg, 0.61 mmol, 0.05 eq.) were added. The flask was flushed with argon and the solution was heated to reflux for 5 hours. After evaporation to dryness, 3.93 g (11.88 mmol, >98%) of the desired compound C was obtained as a colorless solid.

### Example 2

### Synthesis of Boc Protected Compound D

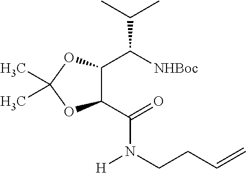

Compound C (1.40 g, 4.23 mmol, 1 eq.) was dissolved in methanol/water (1:1, 20 mL) in a 50 mL flask and a 1 M aq. lithium hydroxide solution (13 mL, 533 mg, 12.69 mmol, 3 eq.) was added at 0° C. The mixture was stirred for further 30 min at room temperature. After evaporation of the methanol, a 20% aq. citric acid solution was added to acidify the reaction mixture. Extraction with dichloromethane (3×50 mL), drying over Na₂SO₄, filtering and concentration to dryness yielded 1.31 g (4.15 mmol, >98%) of the intermediate as a white powder.

This intermediate (1.33 g, 4.20 mmol, 1 eq.), 3-butenylamine hydrochloride (0.54 g, 5.10 mmol, 1.2 eq.), HOAt (858 mg, 6.30 mmol, 1.5 eq.) and PyBop (3.28 g, 6.30 mmol, 1.5 eq.) were dissolved in dichloromethane (5 mL) in a 10 mL flask. N,N-Diisopropylethylamine (1.46 mL, 8.40 mmol, 2 eq.) was added at 0° C. and the resulting mixture was stirred overnight at room temperature. The reaction was stopped by quenching with a 20% aq. citric acid solution and compound D was extracted from the mixture with chloroform (3×50 mL). The combined organic layers were dried over Na₂SO₄, filtered and concentrated to dryness. The crude product was purified by flash column chromatography (20% ethyl acetate in cyclohexane) to afford 1.27 g (3.43 mmol, 82%) of compound D as a colorless solid.

### Example 3

### Synthesis of Boc Protected Compound F

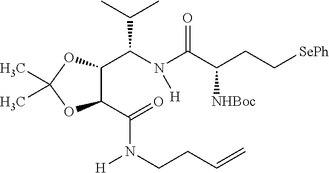

Compound E is prepared as follows: Sodium borohydride (125 mg, 3.3 mmol, 4.4 eq.) was disposed under argon in a 100 mL flame dried flask. A solution of diphenyl diselenide (937 mg, 3.0 mmol, 1 eq.) in dimethylformamide (20 mL) was added, followed by addition of a solution of Boc-homoserine lactone (603 mg, 3.0 mmol, 1 eq.) in dimethylformamide (20 mL). The resulting mixture was heated to 100° C. for 90 minutes. After cooling to 0° C., methanol (5 mL) was added and the mixture was stirred for an hour. The solvents were removed in vacuo and the remaining residue was partitioned between diethyl ether (150 mL) and 100 mM NaOAc buffer (pH 5.0). The aqueous layer was extracted twice more with diethyl ether (150 mL). The combined organic layers were dried over Na₂SO₄, filtered and concentrated. The crude product was purified by flash column chromatography (40% ethyl acetate in cyclohexane) to afford 973 mg (2.72 mmol, 91%) of compound E as a colorless solid.

Compound F is prepared as follows:
Compound D (710 mg, 1.92 mmol, 1 eq.) was dissolved under argon in dichloromethane (2 mL) in a 10 mL flame-dried flask. 2,6-Lutidine (446 μL, 3.84 mmol, 2 eq.) and trimethylsilyl trifluoro methanesulfonate (522 μL, 2.88 mmol, 1.5 eq.) were added and the resulting mixture was stirred for further 15 minutes. The reaction was quenched upon addition of a saturated aq. NH₄Cl solution. The pH of the water phase was adjusted to 9 by addition of a 2 M aq. NaOH solution and was extracted with dichloromethane. The combined organic layers were washed with brine, dried over Na₂SO₄, filtered and concentrated to dryness, yielding 508 mg (1.88 mmol, >98%) of the desired intermediate as a white powder.

This intermediate (512 mg, 1.90 mmol, 1 eq.), compound D (878 mg, 2.45 mmol, 1.3 eq.), PyBop (1.48 g, 2.85 mmol, 1.5 eq.) and HOAt (388 mg, 2.85 mmol, 1.5 eq.) were dissolved in dichloromethane (10 mL) in a 25 mL flask. The solution was cooled to 0° C. and N,N-diisopropylethylamine (662 μL, 3.80 mmol, 2 eq.) was added. The reaction was stirred overnight at room temperature, quenched by addition of a 20% aq. citric acid solution and extracted with chloroform (3×50 mL). The combined organic layers were dried over Na₂SO₄, filtered and concentrated to dryness. The crude product was purified by flash column chromatography (20% ethyl acetate in cyclohexane) to afford 1.03 g (1.69 mmol, 89%) of compound F as a colorless solid.

### Example 4

### Synthesis of Boc Protected Compound G

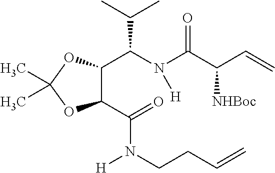

Compound F (925 mg, 2.04 mmol) was dissolved in dichloromethane (85 mL) in a 250 mL flask. Hydrogen peroxide (30% in water, 10 mL) and N,N-diisopropylethylamine (10 mL) were added and the resulting mixture was heated to 50° C. for 3 hours. The reaction was quenched by addition of a saturated aq. CuSO₄ solution. Addition of ethyl acetate (50 mL) and a 10% aq. KHSO₄ solution (50 mL) generated a biphasic mixture which was separated in a funnel. The organic phase was washed with a 5% aq. NaHCO₃ solution (50 mL) and brine (50 mL), dried over Na₂SO₄, filtered and concentrated to dryness. The crude product was purified by flash column chromatography (20% ethyl acetate in cyclohexane) to afford 861 mg (1.90 mmol, 93%) of compound G as a colorless solid.

### Example 5

### Synthesis of Boc Protected Compound H

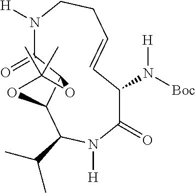

Compound G (737 mg, 1.620 mmol, 1 eq.) was dissolved under argon in toluene (800 mL) in a 1 L flame-dried flask and heated to 90° C. A solution of Grubbs' 2^{nd} generation catalyst (207 mg, 0.243 mmol, 0.15 eq.) in toluene (25 mL) was added over 8 hours with a syringe pump to the preheated mixture. The resulting solution was stirred for further 10 hours at 90° C. After concentration to dryness, the crude product was purified by flash column chromatography (50% ethyl acetate in cyclohexane) to afford 335 mg (0.787 mmol, 49%) of compound H as a light brown solid. The product was pure enough to be used in the next step without further purification. Nevertheless, a second flash column chromatography can be performed to completely eliminate the remaining traces of ruthenium residues.

### Example 6

### Synthesis of CH₃ Protected Compound K

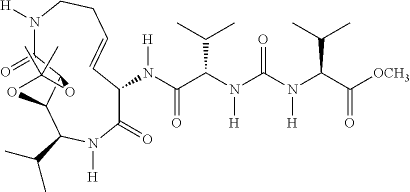

Compound J is prepared as follows:
Methyl (S)-(−)-2-isocyanato-3-methylbutyrate (431 μL, 3.00 mmol, 1 eq.) was dissolved under argon in dichloromethane (10 mL) in a 25 mL flame-dried flask. A solution of tert-butyl valine hydrochloride (629 mg, 3.00 mmol, 1 eq.) and N,N-diisopropylethylamine (1.05 mL, 6.00 mmol, 2 eq.) in dichloromethane (5 mL) was added and the resulting mixture was stirred overnight at room temperature. The reaction was quenched by addition of a 20% aq. citric acid solution and the desired product was extracted with chloroform (2×20 mL). The combined organic layers were dried over Na₂SO₄, filtered and evaporated to dryness. The crude product was purified by flash column chromatography (15% ethyl acetate in cyclohexane) to yield 892 mg (2.70 mmol, 90%) of an intermediate as colorless crystals.

This intermediate (892 mg, 2.70 mmol) was dissolved in formic acid (6 mL) in a 25 mL flask. Some drops of water were added and the mixture was stirred overnight. After concentration to dryness and co-evaporation with toluene, crude compound J was obtained which was purified by flash column chromatography (70% ethyl acetate in cyclohexane) to yield 674 mg (2.46 mmol, 91%) of compound J as a colorless solid.

Compound K is prepared as follows:
Compound H (295 mg, 0.69 mmol, 1 eq.) was dissolved in dichloromethane (4 mL) under argon in a 10 mL flame-dried flask. 2,6-Lutidine (161 μL, 1.38 mmol, 2 eq.) and trimethylsilyl trifluoro methanesulfonate (188 μL, 1.04 mmol, 1.5 eq.) were added at room temperature and the resulting mixture was stirred for 15 minutes. Addition of a saturated aq. NH₄Cl solution quenched the reaction. The pH was adjusted to 9 by addition of a 2 M NaOH solution and the desired product was extracted from the water phase with dichloromethane. The combined organic layers were washed with brine, dried over Na₂SO₄, filtered and concentrated to dryness to yield 221 mg (0.68 mmol, 98%) of the intermediate as a white solid.

This intermediate (161 mg, 495 μmol, 1 eq.), compound J (190 mg, 693 μmol, 1.4 eq.), PyBOP (387 mg, 743 μmol, 1.5 eq.) and HOAt (102 mg, 743 μmol, 1.5 eq.) were dissolved in dichloromethane (10 mL) in a 10 mL flask. The solution was cooled to 0° C. and N,N-diisopropylethylamine (173 μL, 990 μmol, 2 eq.) was added. The reaction was stirred overnight at room temperature, was diluted with methanol/dichloromethane (1:9, 25 mL) and then washed with a 20% aq. citric acid solution and a 5% aq. NaHCO₃ solution. The organic layer was dried over Na₂SO₄, filtered and concentrated to dryness. The crude product was purified by flash column chromatography (5% methanol in ethyl acetate) to yield 273 mg (469 mmol, 95%) of compound K as a colorless solid.

### Example 7

### Synthesis of CH₃ Protected Compound L

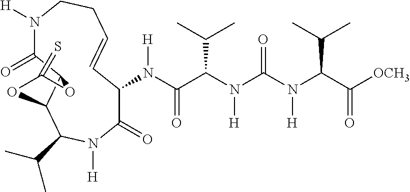

In a 10 mL vessel was placed compound K (5 mg, 8.6 μmol), formic acid/methanol (6:4, 5 mL) and a magnetic stirring bar. The vessel was sealed with a septum, placed into the MW cavity, and locked with the pressure device. Constant microwave irradiation of 45 W as well as a simultaneous air-cooling (300 kPa, 45 Psi) were used during the entire reaction time (90 min, 110° C., resulting reaction pressure 6 bar). After cooling to room temperature, the solvent was removed under reduced pressure to afford 4.5 mg (8.4 μmol, >98%) of the dihydroxyl intermediate as a colorless solid. The product was pure enough to be used in the next step without further purification.

After performing this reaction several times, all product fractions were pooled and the resulting residue of the dihydroxyl derivative (92 mg, 170 μmol, 1 eq.) was dissolved under argon in tetrahydrofurane (50 mL) in a 100 mL flame-dried flask. To this solution was added thiocarbonyl diimidazole (303 mg, 1.70 mmol, 10 eq.) and 4-(dimethylamino)pyridine (208 mg, 1.70 mmol, 10 eq.). The resulting reaction mixture was heated to 80° C. and stirred at this temperature overnight. After recooling to room temperature, a small portion of silica gel was added and the solvent was removed under vacuo. The adsorbed crude product was purified by flash column chromatography (4% methanol in dichloromethane) to yield 88 mg (151 μmol, 89%) of compound L as a colorless solid.

### Example 8

### Synthesis of Syringolin A

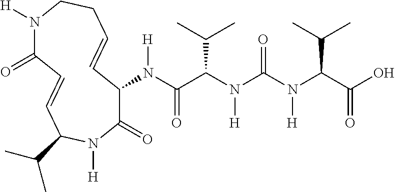

Compound L (20.0 mg, 34 μmol, 1 eq.) was dissolved under argon in trimethyl phosphite (2 mL) in a 10 mL flame-dried flask. The resulting mixture was refluxed for 3 hours at 130° C. After concentration to dryness, the crude product was purified by flash column chromatography (10% methanol in dichloromethane) to yield 13.1 mg (26 μmol, 76%) of an intermediate as a colorless solid.

This intermediate (8.0 mg, 16 μmol, 1 eq.) and aluminium trichloride (17.1 mg, 128 μmol, 8 eq.) were dissolved under argon in ethyl methyl sulfide (400 μL) in a 10 mL flame-dried flask. The resulting mixture was stirred for 1 hour at room temperature. After concentration to dryness, the crude product was purified by flash column chromatography (2% acetic acid+15% methanol in dichloromethane) to yield 7.3 mg (15 μmol, 92%) of syringolin A as a colorless solid.

### Example 9

### Synthesis of Boc and tBu Protected Compound 2

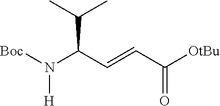

Compound 1 is commercially available from Sigma-Aldrich. Compound 1 (500 mg, 2.16 mmol, 1 eq.) was dissolved in toluene (22 mL) under argon in a 100 mL flame-dried flask. The solution was cooled to −78° C. and a 1 M solution of DIBAL-H in toluene (4.4 mL, 4.32 mmol, 2 eq.) was slowly added over 2 hours. After further 2 hours of stirring, the mixture was quenched with a 1.2 M solution of potassium sodium tartrate (25 mL) and vigorously stirred at room temperature for further 2 hours. The resulting mixture was extracted with dichloromethane and the organic layers were dried with Na₂SO₄. The solution was filtered and concentrated to give N-(tert-butoxycarbonyl)-(L)-valinal which was directly used in the next step without further purification.

Crude N-(tert-butoxycarbonyl)-(L)-valinal was dissolved in dichloromethane (22 mL) and (tert-butoxycarbonylmethylene)triphenylphosphorane (1.21 g, 3.24 mmol, 1.5 eq.) was added in one portion. After 12 hours of stirring the mixture was concentrated and purified by flash column chromatography (10% ethyl acetate in cyclohexane) to afford 466 mg (1.81 mmol, 84%) of compound 2 as colorless crystals.

### Example 10

### Synthesis of Boc, Troc and tBu Protected Compound 3

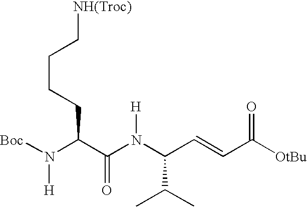

Boc-Lys-OH (3.00 g, 12.18 mmol, 1 eq.) and Na₂CO₃ (1.30 g, 12.18 mmol, 1 eq.) were dissolved in water/dioxane/acetonitrile (19:14:12, 450 mL) in a 1 L flask. The solution was cooled to 0° C. and a solution of 2,2,2-trichloroethyl chloroformate (1.8 mL, 13.40 mmol, 1.1 eq.) in dioxane (160 mL) was slowly added. The resulting mixture was stirred overnight at room temperature, concentrated to dryness and redissolved in a saturated aqueous solution of ammoniumchloride. A crude intermediate was extracted from the aqueous phase with dichloromethane (3×200 mL), dried over Na₂SO₄, filtered and evaporated to dryness. The crude product was purified by flash column chromatography (dichloromethane/methanol/acetic acid=38:1:1) to yield 4.19 g (9.91 mmol, 82%) pure intermediate as a colorless solid.

Compound 2 (1.07 g, 3.57 mmol, 1 eq.) was dissolved under argon in tert-butyl acetate (12 mL, dried over 4 Å molecular sieves) in a 100 mL flame-dried flask. The resulting solution was cooled to −5° C., a 4 M solution of HCl in dioxane (12 mL) was slowly added and the resulting mixture was stirred overnight at 10° C. Evaporation to dryness provided the crude hydrogenchloride salt which was subsequently recrystallized in cyclohexane. The crystals were filtered and washed with small portions of cyclohexane, redissolved in saturated Na₂CO₃ solution and the free amine was extracted from the aqueous phase with dichloromethane. The organic layer was dried over Na₂SO₄, filtered and concentrated to dryness to give pure intermediate-2 in 596 mg (2.99 mmol, 84%) yield.

The lysine-based intermediate (484 mg, 2.43 mmol, 1 eq.) was dissolved in dichloromethane (2 mL) in a 25 mL flask and cooled to 0° C. A solution of intermediate-2 (1.74 g, 4.13 mmol, 1.7 eq.), PyBop (3.80 g, 7.30 mmol, 3 eq.), HOAt (994 mg, 7.30 mmol, 3 eq.) and N,N-diisopropylethylamine (2.65 mL, 14.60 mmol, 6 eq.) in dichloromethane (8 mL) was added and stirred overnight at room temperature. After evaporation to dryness, the crude product was purified by flash column chromatography (30% ethyl acetate in cyclohexane) to yield 1.25 g (2.07 mmol, 85%) of compound 3 as a colorless solid.

### Example 11

### Synthesis of Fm Protected Compound 4

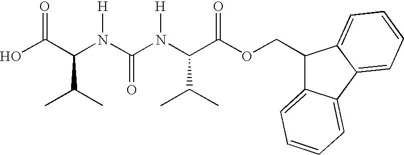

Boc-valine-OFm (3.96 g, 10.0 mmol, 1 eq., prepared according to B. Henkel, L. Zhang, E. Bayer, Liebigs Annalen/Recueil 1997, 10, 2161-2168) was dissolved in dichloromethane (75 mL) in a 250 mL flask and trifluoroacetic acid (25 mL) was slowly added. The mixture was stirred for 30 minutes, followed by evaporation to dryness. Addition of toluene and re-evaporation to dryness yielded 4.10 g (10.0 mmol, >98%) of an intermediate as a colorless solid.

Triphosgene (110 mg, 0.37 mmol, 1.11 eq.) was dissolved under argon in dichloromethane (2 mL) in a 25 mL flame-dried flask and a solution of commercially available valine tert-butylester hydrochloride (210 mg, 1.00 mmol, 1.00 eq.) and N,N-diisopropylethylamine (385 μL, 2.20 mmol, 2.20 eq.) in dichloromethane (3.5 mL) was added over 30 minutes. The mixture was stirred for further five minutes, then a mixture of the intermediate (410 mg, 1.00 mmol, 1.00 eq.) and N,N-diisopropylethylamine (385 μL, 2.20 mmol, 2.20 eq.) in dichloromethane (2.0 mL) was added in one portion. The resulting mixture was stirred for 10 minutes, concentrated to dryness, the residue was redissolved in ethyl acetate and successively washed with a 10% aq. KHSO₄ solution, a 5% aq. NaHCO₃ solution and with brine. The organic layer was dried over Na₂SO₄, filtered and evaporated to dryness. The resulting crude product was purified by flash column chromatography (15% ethyl acetate in cyclohexane) to yield 299 mg (0.61 mmol, 61%) of an additional intermediate as white crystals.

This intermediate (299 mg, 0.61 mmol) was dissolved in formic acid (4 mL) in a 25 mL flask. Some drops of water were added and the mixture was stirred overnight. Evaporation to dryness, addition of toluene and re-evaporation yielded 262 mg (0.60 mmol, >98%) of compound 4 as a colorless solid.

### Example 12

### Synthesis of Fm, Troc and tBu Protected Compound 5

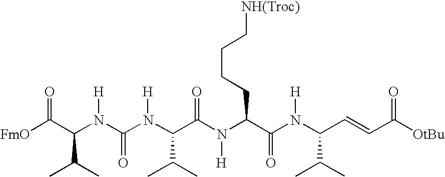

Compound 3 (935 mg, 1.55 mmol, 1 eq.) was dissolved under argon in tert-butyl acetate (12 mL, dried over 4 Å molecular sieves) in a 100 mL flame-dried flask and cooled to −5° C. A solution of 4 M HCl in dioxane (12 mL) was slowly added and stirred overnight at 10° C. The resulting mixture was evaporated to dryness and recrystallized in cyclohexane. The crystals were filtered, washed with small portions of cyclohexane and redissolved in saturated aqueous Na₂CO₃ solution. The free amine was extracted from the aqueous phase with ethyl acetate (3×50 mL), dried over Na₂SO₄, filtered and concentrated to dryness to give 628 mg (1.29 mmol, 83%) of an intermediate as a colorless oil.

This intermediate (29 mg, 58 μmol, 1 eq.) was dissolved in dichloromethane (1 mL) in a 10 mL flask and cooled to 0° C. A solution of compound 4 (31 mg, 69 μmol, 1.2 eq.), PyBop (46 mg, 87 μmol, 1.5 eq.), HOAt (12 mg, 87 μmol, 1.5 eq.) and N,N-diisopropylethylamine (32 μL, 180 μmol, 3 eq.) in dichloromethane (1 mL) were added and the resulting mixture was stirred overnight at room temperature. After evaporation, the crude product was purified by flash column chromatography (60% ethyl acetate in cyclohexane) to yield 40 mg (43 μmol, 75%) of compound 5 as a colorless solid.

### Example 13

### Synthesis of Fm Protected Compound 6

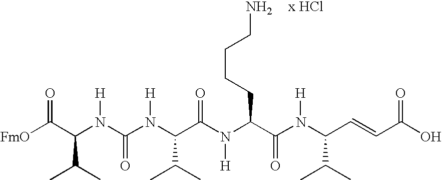

Compound 5 (60 mg, 65 μmol, 1 eq.) was dissolved in tetrahydrofurane (2 mL) in a 10 mL flask. Acetic acid was added (2 mL), followed by zinc powder (638 mg, 9.76 mmol, 150 eq.) which was added in portions over 30 minutes. After 3 hours of vigorous stirring, the mixture was filtered over a small plug of Celite and washed with ethyl acetate. After evaporation to dryness, 47 mg (63 μmol, 97%) of the deprotected amine was obtained which was used in the next step without further purification.

The cleaved intermediate (47 mg, 63 μmol) was dissolved in formic acid (4 mL) in a 10 mL flask and some drops of water were added. The resulting mixture was stirred overnight, concentrated to dryness, redissolved in diluted aq. HCl and re-evaporated to dryness. Addition of toluene and concentration to dryness yielded 45 mg (62 μmol, >98%) of compound 6 as a colorless solid.

### Example 14

### Synthesis of Syringolin B

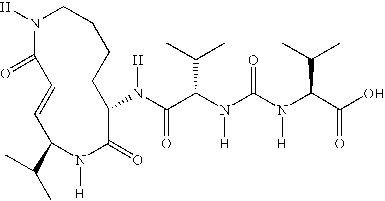

PyBOP (339 mg, 651 μmol, 3 eq.), HOAt (89 mg, 651 μmol, 3 eq.) and N,N-diisopropylethylamine (114 μL, 651 μmol, 3 eq.) were dissolved under argon in dimethylformamide (114 mL) in a 500 mL flame-dried flask. A solution of compound 6 (150 mg, 217 μmol, 1 eq.) and N,N-diisopropylethylamine (114 μL, 651 μmol, 3 eq.) in N,N-dimethylformamide (58 mL) was slowly added over 8 hours with a syringe pump and stirred for further 24 hours. The reaction was quenched by addition of a 20% aq. citric acid solution and extracted with ethyl acetate. The organic layers were washed with water (2×50 mL) and dried over Na₂SO₄, filtered and evaporated to dryness. The remaining residue was purified by flash column chromatography (4% methanol in ethyl acetate) to yield 44 mg (65 μmol, 30%) of the cyclized product.

The cyclized product (7.70 mg, 11.4 μmol, 1 eq.) was dissolved in N,N-dimethylformamide (800 μL) in a 10 mL flask and piperidine (200 μL) was added. The mixture was stirred for one hour and then evaporated to dryness. The remaining residue was purified by preparative HPLC (using H₂O with 0.1% TFA (solvent A) and acetonitrile with 0.1% TFA (solvent B) at a flow of 25 mL/min. Gradient: from 0 to 10 min: 90% solvent A/10% solvent B; from 10 to 30 min: from 90% solvent A/10% solvent B to 70% solvent A/30% solvent B; from 30 to 50 min: from 70% solvent A/30% solvent B to 40% solvent A/60% solvent B; from 50 to 60 min: from 40% solvent A/60% solvent B to 0% solvent A/100% solvent B; from 60 to 80 min: 0% solvent A/100% solvent B) to yield mg (8.3 μmol, 73%) of Syringolin B as a colorless powder.

1. Method for synthesizing syringolin A comprising the steps: reacting compound G 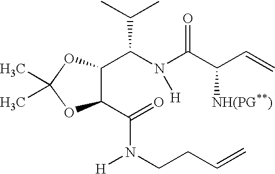
wherein PG** represents a suitable amino protecting group,
with Grupps II catalyst in order to obtain compound H 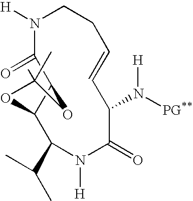
which is reacted with compound J 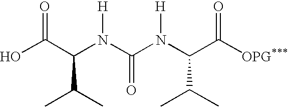
wherein PG*** represents a suitable carboxy protecting group, in order to obtain compound K 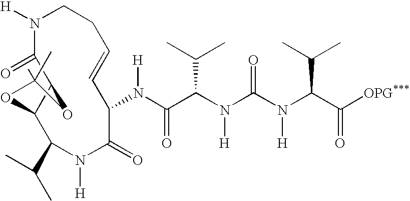
then the ketal group is cleaved and the vicinal dihydroxy group is converted into a carbon carbon double bond followed by a final deprotection step of the carboxyl protecting group in order to obtain syringolin A.

2. Method for synthesizing syringolin B comprising the steps: reacting compound 2 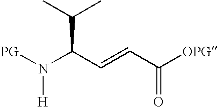
wherein PG and PG″ represent suitable amino or carboxy protecting groups, with a protected amino acid lysine of the formula PG′″-Lys(PG″″)-OH, wherein PG′″ and PG″″ represent suitable amino protecting groups in order to obtain compound 3 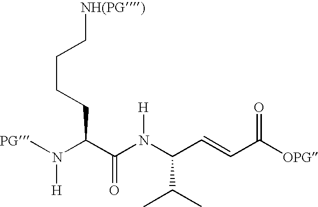
which is further reacted with compound 4 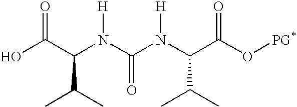
wherein PG* is a suitable carboxy protecting group in order to generate compound 5 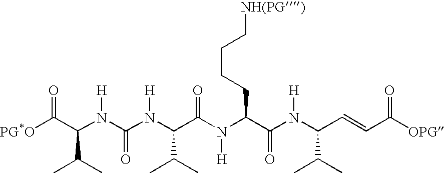
which is deprotected to form compound 6 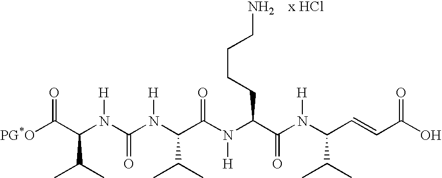
and compound 6 is subjected to macrolactamisation conditions and after a last deprotection step the final product syringolin B is obtained [chemical structure]
